(19) United States
(12) Patent Application Publication
(10) Pub. No.: US 2021/0362320 A1
(43) Pub. Date: Nov. 25, 2021

(54) MOBILE EPIDEMIC PREVENTION AND DISINFECTION ROBOT AND ITS WORKING METHOD

(71) Applicant: HONG FU JIN PRECISION INDUSTRY (ShenZhen) CO., LTD., Shenzhen (CN)

(72) Inventor: FU-HSIUNG YANG, New Taipei (TW)

(21) Appl. No.: 16/882,294

(22) Filed: May 22, 2020

(30) Foreign Application Priority Data
May 21, 2020 (TW) 109116827

(51) Int. Cl. B25J 5/00; B25J 9/00; B25J 11/00; B25J 9/16; G05B 19/4155; G05D 1/02

(52) U.S. Cl. CPC B25J 5/00; B25J 9/0087; B25J 11/0085; G05B 2219/45098; G05B 19/4155; G05D 1/0212; G05D 1/0255; B25J 9/1694

(57) ABSTRACT

A mobile epidemic prevention and disinfection robot includes a body and a control system installed on the body. The body includes a mobile base, a dust collection module, a disinfection system, a temperature detection system, and a delivery system. The delivery system includes two robot arms and a storage box. The control system includes a control module, a path planning module, and an obstacle dodge module. The control module is used to control the movement of the mobile epidemic prevention and disinfection robot, and the two robot arms to perform corresponding actions. The path planning module plans the optimal movement information of the mobile epidemic prevention and disinfection robot from the current position to a destination according to a three-dimensional map. The obstacle dodge module controls the mobile epidemic prevention and disinfection robot to dodge obstacles.

### CROSS-REFERENCE TO RELATED APPLICATIONS

This application claims all benefits accruing under 35 U.S.C. § 119 from Taiwan Patent Application No. 109116827, filed on May 21, 2020, in the Taiwan Intellectual Property Office, the contents of which are hereby incorporated by reference.

### FIELD

The subject matter herein generally relates to a mobile epidemic prevention and disinfection robot and its working method.

### BACKGROUND

According to the statistics, about 4.5% of inpatients died from medical care related infections. The average annual cost to deal with the complications caused by the infections is about US $36.5 billion, which not only aggravates the diseases, but also increases the medical expenses. In addition, hospitals, factories, schools, and stores, which have larger areas, may require more manpower costs and time on traditional manual cleaning and disinfection methods, and the quality of disinfection is unstable.

What is needed, therefore, is to provide a mobile epidemic prevention and disinfection robot.

### BRIEF DESCRIPTION OF THE DRAWINGS

Many aspects of the embodiments can be better understood with reference to the following drawings. The components in the drawings are not necessarily drawn to scale, the emphasis instead being placed upon clearly illustrating the principles of the embodiments. Moreover, in the drawings, like reference numerals designate corresponding parts throughout the several views.

FIG. 1 is a schematic view of a first embodiment of a mobile epidemic prevention and disinfection robot.

FIG. 2 is a schematic view of the first embodiment of a carrier system of the mobile epidemic prevention and disinfection robot of FIG. 1.

FIG. 3 is a process flow of a working method of the mobile epidemic prevention and disinfection robot of FIG. 1.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein can be practiced without these specific details. In other instances, methods, procedures and components have not been described in detail so as not to obscure the related relevant feature being described. The drawings are not necessarily to scale and the proportions of certain parts may be exaggerated to better illustrate details and features. The description is not to be considered as limiting the scope of the embodiments described herein.

Several definitions that apply throughout this disclosure will now be presented.

The connection can be such that the objects are permanently connected or releasable connected. The term “substantially” is defined to be essentially conforming to the particular dimension, shape or other word that substantially modifies, such that the component need not be exact. The term “comprising” means “including, but not necessarily limited to”; it specifically indicates open-ended inclusion or membership in a so-described combination, group, series and the like. It should be noted that references to “an” or “one” embodiment in this disclosure are not necessarily to the same embodiment, and such references mean at least one.

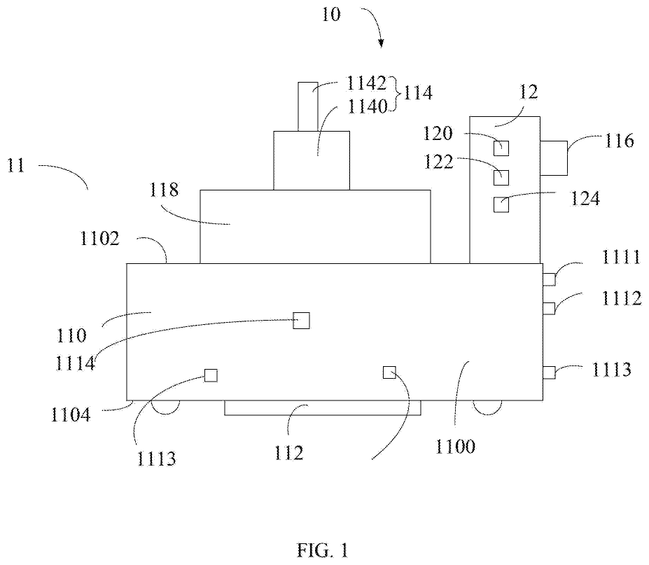

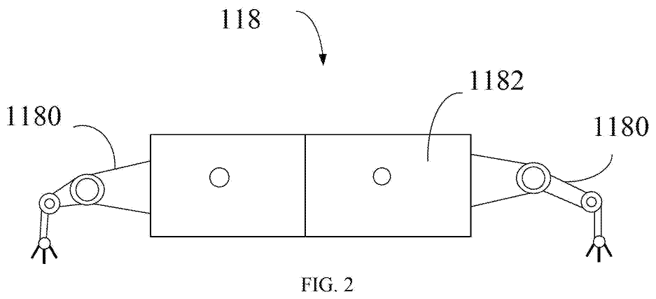

Referring to FIG. 1 and FIG. 2, a mobile epidemic prevention and disinfection robot 10 of one embodiment includes a body 11 and a control system 12. The control system 12 is located in the body 11 and used for controlling the body 11.

The body 11 includes a mobile base 110, a dust collection module 112, a disinfection system 114, a temperature detection system 116, and a delivery system 118.

The mobile base 110 includes a base 1100, an image acquisition module 1111, a laser detection module 1112, a sensing module 1113, and a data storage module 1114 located on the base 1100. The image acquisition module 1111 has a stereo camera. The stereo camera is used for photographing an image of objects and humans in front of the mobile epidemic prevention and disinfection robot 10 in the process of moving to a destination, so that an image signal is formed. The image signal is converted into a digital signal. By comparing the digital signal with a pre-stored digital signal in the database, the characteristics of the object and the human can be identified. The data storage module 1114 stores a map of an area to be moved, a corresponding map description file, data information of commonly used objects of a hospital, location information of waste recycling, image information of common objects and people, and so on. The laser detection module 1112 has a laser radar. The laser radar can scan the surrounding environment and establish a three-dimensional map. The sensing module 1113 includes an ultrasonic sensor, an infrared sensor, or other sensors that can detect a distance between objects. The sensing module 1113 is used to sense obstacles or a target position, and a distance between the current position of the mobile epidemic prevention and disinfection robot 10 and the obstacles. Furthermore, the sensing module 1113 can identify objects in the environment to avoid the obstacles (slow down, turn, stop) and reach the designated area. The mobile base 110 includes a first surface 1102 and a second surface 1104 opposite to the first surface 1102, the second surface 1104 is close to the ground, and the first surface 1102 is away from the ground.

The dust collection module 112 is located on the second surface 1104 of the mobile base 110, and the disinfection system 114 is located on the first surface 1102 of the mobile base 110. The temperature detection system 116 is used for detecting human body temperatures. The temperature detection system 116 includes a network infrared imaging temperature camera. The network infrared imaging temperature camera automatically measures the temperature of a human body after detecting the human body. When the temperature detection system 116 detects an abnormal temperature, it can send a message to a nursing staff. The disinfection system 114 includes a spray device 1140 and an ultraviolet lamp 1142 located on the spray device 1140. The delivery system 118 is used for transferring objects. The delivery system 118 can be used to transport the objects such as sweat clothes and consumables after operations, so as to avoid further infections of cleaning person by these objects. The delivery system 118 includes two robot arms 1180 and a storage box 1182. The two robot arms 1180 are installed on two opposite sides of the storage box 1182 respectively. The storage box 1182 is used for placing the objects grabbed by the two robot arms 1180. The delivery system 118 can be located on the first surface 1102 of the mobile base 110 side by side with the disinfection system 114. The delivery system 118 and the disinfection system 114 can also be vertically and sequentially arranged on the first surface 1102 of the mobile base 110.

The control system 12 is mounted on the first surface 1102 of the mobile base 110. The control system 12 includes a control module 120, a path planning module 122, and an obstacle dodge module 124. According to the collected information, the control module 120 transmits instructions for controlling the movement of the mobile base 110, and the two robot arms 1180 to perform corresponding actions, such as taking and placing the objects. The control module 120 is also provided with a disinfection management program for controlling the turning on and turning off of the disinfection system 114, a spraying speed of a disinfectant, a moving path and a disinfection time. According to the three-dimensional map, the path planning module 122 can plan the optimal mobile information of the mobile epidemic prevention and disinfection robot 10 from the current position to the destination, and the obstacle dodge module 124 can control the mobile epidemic prevention and disinfection robot 10 to dodge the obstacles.

In use, a person can realize the remote operation of the mobile epidemic prevention and disinfection robot 10 through the connection between the App and the mobile epidemic prevention and disinfection robot 10.

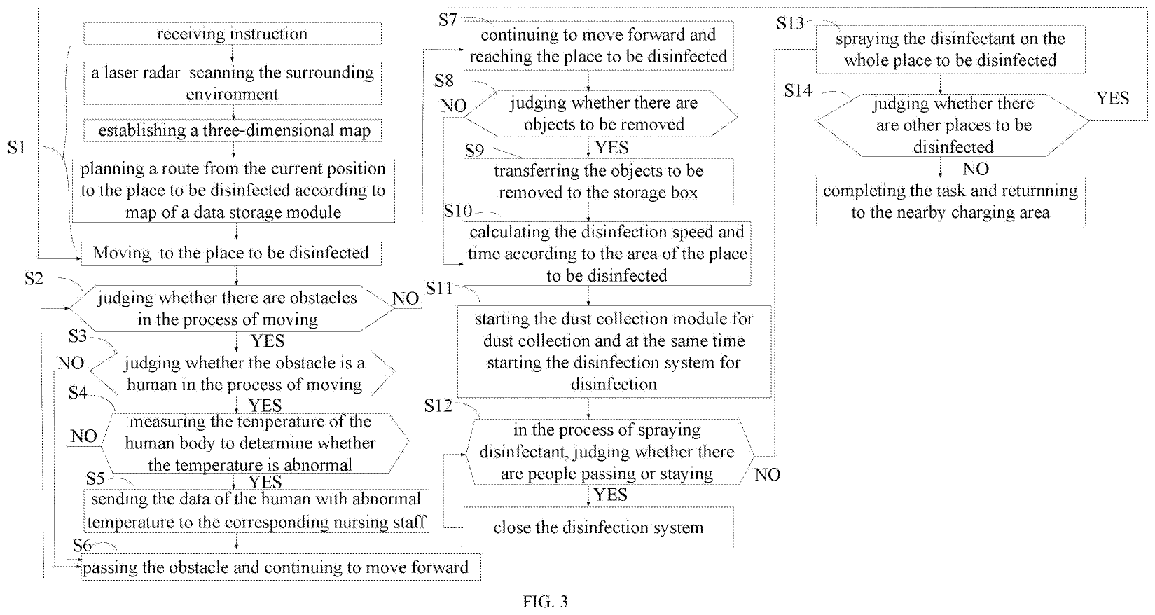

Referring to FIG. 3, a working method of the mobile epidemic prevention and disinfection robot 10 of one embodiment is provided. The method includes the following steps:

Step 1: receiving location and quantity information of a place to be disinfected by the mobile epidemic prevention and disinfection robot 10, planning a mobile route, and moving to the place to be disinfected, and then enter step 2;

Step 2: judging whether there are obstacles in the process of the movement of the mobile epidemic prevention and disinfection robot 10, if yes, enter step 3, if not, enter step 7;

Step 3: judging whether the obstacle is a human in the process of the movement of the mobile epidemic prevention and disinfection robot 10, if yes, enter step 4, if not, enter step 6;

Step 4: measuring the temperature of the human body to determine whether the temperature is abnormal, if yes, enter step 5, if not, enter step 6;

Step 5: sending the data of the human with abnormal temperature to a corresponding nursing staff, and then going to step 6;

Step 6: passing the obstacle and continuing to move forward, then returning to step 2;

Step 7: continuing to move forward and reaching the place to be disinfected, and then going to step 8;

Step 8: judging whether there are objects to be removed, if yes, enter step 9, if not, enter step 10;

Step 9: transferring the objects to be removed to the storage box 1182, and then going to step 10;

Step 10: calculating the disinfection speed and disinfection time according to the area of the place to be disinfected, and then going to step 11;

Step 11: starting the dust collection module 112 for collecting dust, and at the same time starting the disinfection system 114 for disinfecting, and then going to step 12;

Step 12: judging whether there are people passing or staying in the process of spraying disinfectant, if yes, turn off the disinfection system 114, and then repeat step 12, if not, enter step 13;

Step 13: spraying the disinfectant on the whole place to be disinfected, and then going to step 14; and

Step 14: judging whether there are other places to be disinfected, if yes, return to step 1, if not, return to a nearby charging area.

In step 1, the laser radar of the mobile epidemic prevention and disinfection robot 10 scans the surrounding environment and establishes a three-dimensional map. The path planning module 122 compares the three-dimensional map with the map of the area to be moved in the data storage module 1114, and automatically plans the route from the current position to the place to be disinfected. Then the control system 12 controls the mobile epidemic prevention and disinfection robot 10 to move to the place to be disinfected according to the planned route.

In step 2, in detail, in the process of moving, the sensing module 1113 senses whether there is an obstacle in real time. If an obstacle is encountered, the stereo camera takes pictures of the obstacle.

In step 3, according to analyzing the image taken by the stereo camera, it is determined whether the obstacle is a human.

In step 4, the network infrared imaging temperature measuring camera measures the human body temperature, obtains the temperature information of the human body, and analyzes the temperature. In one embodiment, the abnormal temperature is higher than 38° C.

In step 6, when the sensing module 1113 detects an obstacle, the obstacle dodge module 124 will start the dodge function to automatically avoid the obstacle. Not only in the moving process, but also in all subsequent moving processes, the sensing module 1113 and the obstacle dodge module 124 are all in working state.

In step 8, after arriving at the place to be disinfected, the mobile epidemic prevention and disinfection robot 10 uses the stereo camera to take the image of environment of the place to be disinfected, and then analyzes and judges whether there are any objects to be removed in the place to be disinfected. In one embodiment, the objects to be removed include sweat clothes, consumables, and other objects.

In step 9, the control module 120 gives instructions to the two robot arms 1180 to grab the objects to be removed, and place the objects to be removed in the storage box 1182. Then, according to the types of objects stored in the data storage module 1114 and the recovery location, the control module 120 determines where to transport the objects to be removed and what to do with them.

In step 11, the disinfection system 114 includes the spray device 1140 and the ultraviolet lamp 1142. Spray disinfection or ultraviolet disinfection can be selected as needed. According to the calculated speed and calculated time, the control system 12 controls the moving speed of the mobile base 110, the quantity and speed of the sprayed disinfectant. The dust collection module 112 is located on the second surface 1104 of the mobile base 110, so that dust on the ground can be absorbed. The spray device 1140 located on the first surface 1102 of the mobile base 110 is used for spraying a disinfectant solution while the dust absorption module 112 absorbs dust.

In step 12, in the process of spraying the disinfectant, if the sensing module 1113 and the stereo camera detect that there is a person passing by, the control module 120 issues a command to turn off the disinfection system 114 until the person leaves. The control module 120 issues a command to turn on the disinfection system 114, which can avoid injury to the person.

In step 14, according to the information of the place to be disinfected stored in the data storage module 1114, the mobile epidemic prevention and disinfection robot 10 judges whether there are other places to be disinfected. If there are other places to be disinfected, the mobile epidemic prevention and disinfection robot 10 needs to return to step 1 and do the same action again. If there are no other places to be disinfected, the task is complete and the mobile epidemic prevention and disinfection robot 10 needs to be charged in the nearest charging area.

The disinfection system 114 can include the ultraviolet lamp 1142. The mobile epidemic prevention disinfection robot 10 chooses the spray device 1140 or the ultraviolet lamp 1142 for disinfection according to needs. When the ultraviolet lamp 1142 is used for disinfecting, the control system 12 can calculate the disinfection time according to an area of the place to be disinfected, and carry out 360 degree disinfection around the environment independently, so as to solve the problem that a fixed ultraviolet sterilizer cannot effectively disinfect all areas in the environment. When the mobile epidemic prevention disinfection robot 10 detects the human, the ultraviolet lamp 1142 is turned off to avoid the injury caused by the human body's long-term contact with ultraviolet.

The mobile epidemic prevention and disinfection robot 10 can realize automatic disinfection, automatically avoid person after detecting them, and find out the abnormal person and inform the nursing staff to handle them in time. In addition, the delivery system can automatically identify and transport objects such as sweat clothes and consumables after operations, to avoid further infections of the cleaning person. In addition, the remote operation of the mobile epidemic prevention and disinfection robot 10 is realized by connecting the App and the mobile epidemic prevention and disinfection robot 10. In addition, the method of using the mobile epidemic prevention and disinfection robot 10 to receive and deliver objects completely replaces the manpower, and saves the manpower and time.

The embodiments shown and described above are only examples. Even though numerous characteristics and advantages of the present technology have been set forth in the forego description, together with details of the structure and function of the present disclosure, the disclosure is illustrative only, and changes may be made in the detail, including in matters of shape, size and arrangement of the parts within the principles of the present disclosure up to, and including, the full extent established by the broad general meaning of the terms used in the claims.

Depending on the embodiment, certain of the steps of methods described may be removed, others may be added, and the sequence of steps may be altered. The description and the claims drawn to a method may include some indication in reference to certain steps. However, the indication used is only to be viewed for identification purposes and not as a suggestion as to an order for the steps.

What is claimed is:

1. A mobile epidemic prevention and disinfection robot comprising:
a body, wherein the body comprises:
a mobile base having a first surface and a second surface opposite to the first surface;
a dust collection module located on the second surface of the mobile base;
a disinfection system located on the first surface of the mobile base;
a temperature detection system configured to measure a body temperature of a human; and
a delivery system comprising a two robot arms and a storage box, wherein the two robot arms are located on two opposite sides of the storage box separately; and
a control system located on the first surface of the mobile base, wherein the control system comprises:
a control module configured to control a movement of the mobile base and the two robot arms to perform corresponding actions;
a path planning module configured to plan optimal mobile information of the mobile epidemic prevention and disinfection robot from a current position to a destination according to a three-dimensional map; and
an obstacle dodge module configured to control the mobile epidemic prevention and disinfection robot to dodge obstacles.

2. The mobile epidemic prevention and disinfection robot of claim 1, wherein the mobile base comprises:
a base;
an image acquisition module arranged on the base, wherein the image acquisition module is used for photographing an image of objects and humans in front of the mobile epidemic prevention and disinfection robot in front of the mobile epidemic prevention and disinfection robot in a process of moving to the destination to form an image signal, and identifying characteristics of the objects and the human image;
a data storage module arranged on the base, wherein the data storage module stores a map of an area to be moved, a corresponding map description file, data information of commonly used objects in a hospital, and location information of waste recycling;
a laser detection module arranged on the base, wherein the laser detection module has a laser radar, which scans a surrounding environment and establishes the three-dimensional map; and
a sensing module arranged on the base, wherein the sensing module is used to sense the obstacles or a target position and a distance between current position of the mobile epidemic prevention and disinfection robot and the obstacles.

3. The mobile epidemic prevention and disinfection robot of claim 2, wherein the sensing module comprises an ultrasonic sensor or an infrared sensor.

4. The mobile epidemic prevention and disinfection robot of claim 1, wherein the disinfection system comprises a spray device and an ultraviolet lamp.

5. The mobile epidemic prevention and disinfection robot of claim 1, wherein the temperature detection system comprises a network infrared imaging temperature camera, the network infrared imaging temperature camera is configured to automatically measure the body temperature of the human, and send a message to a nursing staff after detecting an abnormal temperature.

6. A working method of a mobile epidemic prevention and disinfection robot, the method comprising:
Step 1: receiving location and quantity information of a place to be disinfected by the mobile epidemic prevention and disinfection robot, planning a mobile route, and moving to the place to be disinfected;
Step 2: judging whether there are obstacles in a process of a movement of the mobile epidemic prevention and disinfection robot, if yes, entering step 3, if no, entering step 7;
Step 3: judging whether the obstacle is a human in the process of the movement of the mobile epidemic prevention and disinfection robot, if yes, entering step 4, if no, entering step 6;
Step 4: measuring a body temperature of the human to determine whether the temperature is abnormal, if yes, entering step 5, if no, entering step 6;
Step 5: sending data of the human with abnormal temperature to a corresponding nursing staff, and then going to step 6;
Step 6: passing the obstacle and continuing to move forward, then returning to step 2;
Step 7: continuing to move forward and reaching the place to be disinfected, and then going to step 8;
Step 8: judging whether there are objects to be removed, if yes, entering step 9, if no, entering step 10;
Step 9: transferring the objects to be removed to a storage box by two body arms, and then going to step 10;
Step 10: calculating a disinfection speed and disinfection time according to an area of the place to be disinfected, and then going to step 11;
Step 11: starting a dust collection module for collecting dust, and at the same time starting a disinfection system for disinfecting, and then going to step 12;
Step 12: judging whether there are people passing or staying in a process of spraying disinfectant, if yes, close the disinfection system, and then repeating step 12, if no, entering step 13;
Step 13: spraying a disinfectant on the whole place to be disinfected, and then going to step 14;
Step 14: judging whether there are other places to be disinfected, if yes, returning to step 1, if no, returning to a nearby charging area.

7. The method of claim 6, wherein the step 1 comprises:
scanning a surrounding environment by a laser radar to establish a three-dimensional map;
comparing the three-dimensional map with a map of an area to be moved in a storage module, and automatically planning the route from a current position to the place to be disinfected by a path planning module; and
controlling the mobile epidemic prevention and disinfection robot to move to the place to be disinfected according to the planned route by a control module.

8. The method of claim 6, wherein the judging whether there are objects to be removed in the step 8 comprises:
taking an image of a target by a stereo camera; and
analyzing and judging whether there are any objects to be removed in the place to be disinfected.

9. The method of claim 8, wherein the objects to be removed comprise sweat clothes and consumables.

10. The method of claim 6, wherein the transferring the objects to be removed to the storage box in the step 9 comprises:
a control module controlling the two robot arms to grab the objects to be removed after receiving an instruction, and
placing the objects to be removed in the storage box, according to types information of objects stored in a data storage module and the recovery location, the control module determining where to transport the objects to be removed and what to do with them.

11. The method of claim 6, wherein the step 12 comprises:
in the process of spraying the disinfectant, if a sensing module and a stereo camera detect that there is a person passing by, issuing a command by the control module to turn off the disinfection system until the person leaves.
